(19) United States
(12) Patent Application Publication
(10) Pub. No.: US 2022/0273646 A1
(43) Pub. Date: Sep. 1, 2022

(54) PHARMACEUTICAL COMPOSITION OF TEMOZOLOMIDE

(71) Applicant: INTAS PHARMACEUTICALS LTD., Thaltej, Gujarat Ahmedabad (IN)

(72) Inventors: Jwalant Vijaybhai DESAI, Sanand Ahmedabad, Gujarat (IN); Mayank SAXENA, Sanand Ahmedabad, Gujarat (IN); Ashutosh JAMLOKI, Sanand Ahmedabad, Gujarat (IN); Venkataramana NAIDU, Sanand Ahmedabad, Gujarat (IN)

(73) Assignee: INTAS PHARMACEUTICALS LTD., Thaltej, Gujarat Ahmedabad (IN)

(21) Appl. No.: 17/627,042

(22) PCT Filed: Jul. 16, 2020
(86) PCT No.: PCT/IB2020/056679

§ 371 Date: Jan. 13, 2022

(30) Foreign Application Priority Data
Jul. 16, 2019 (IN) 201921028629

(51) Int. Cl. A61K 31/495; A61K 47/12; A61K 9/00; A61K 47/10; A61K 47/02

(52) U.S. Cl. CPC A61K 31/495; A61K 47/12; A61K 9/0095; A61K 47/10; A61K 47/02

(57) ABSTRACT

A stable pharmaceutical composition of temozolomide for oral administration. The pharmaceutical composition is in the form of powder for oral suspension, wherein the said powder is reconstituted with a liquid vehicle just before administration. Further the invention relates to process for the preparation of said pharmaceutical composition and its packaging in individual doses.

### RELATED APPLICATIONS

This application is related to Indian Provisional Application No. 201921028629 filed on 16 Jul. 2019 and is incorporated herein in its entirety.

### FIELD OF THE INVENTION

The present invention relates to the stable pharmaceutical composition of temozolomide for oral administration. The said pharmaceutical composition is in the form of powder for oral suspension, wherein the said powder is reconstituted with a liquid vehicle just before administration. Further, the invention relates to process for the preparation of said pharmaceutical composition and its use thereof.

### BACKGROUND OF THE INVENTION

Temozolomide is an alkylating agent and is chemically known as 4-methyl-5-oxo-2,3,4,6,8-pentazabicyclo[4.3.0] nona-2,7,9-triene-9-carboxamide. The empirical formula of Temozolomide is C₆H₆N₆O₂ and the molecular weight is 194.151 g/mol. The chemical structure of Temozolomide is as shown below:

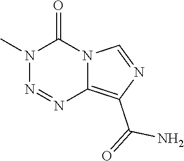

Currently, Temozolomide capsules and powder for intravenous injections are available under the brand name of Temodar® in the US. Commonly, Temozolomide doses are between 5 mg to 250 mg in oral and parenteral routes for adult patients.

Temozolomide is used for the treatment of newly diagnosed glioblastoma multiforme and Refractory Anaplastic Astrocytoma. Cancer is known to be a disease that reduces the quality of life of the patients due to the course of the disease, symptoms and complications; and also due to hard treatment methods and adverse effects resulting from the treatment. Therefore, increasing patient compliance to the treatment in both physical and psychological ways is crucial for the success of the treatment. For instance, the patient may have difficulty in swallowing due to psychological causes, age, symptoms of the disease or adverse effects resulting from the treatment; and may not be able to continue the treatment properly. Further, Temozolomide is cytotoxic drug which require specific precaution during its exposure. Non-compliance between the patient and the treatment significantly affects the success of the treatment of which each stage has vital importance.

The U.S. Pat. No. 5,260,291 discloses the Temozolomide molecule and its use in the treatment of malignant neoplasms including glioma.

WO2018/167627A1 discloses, in general, pharmaceutical composition for oral suspension comprising alkylating antineoplastic agent and pharmaceutically acceptable excipients with improved stability. Notably, temozolomide undergoes hydrolytic degradation, hence an oral suspension as disclosed in the said PCT patent may have adverse effect on stability due to increased hydrolytic degradation.

Particularly, no stability data is provided in the said PCT patent.

Trisselel. al. determined the pharmaceutical acceptability and chemical stability of temozolomide in two extemporaneously compounded suspension formulations prepared from the capsules. Temozolomide extemporaneously prepared as oral suspensions from capsules found chemically stable for at least 60 days at 4° C.

Nygren et. al. had investigated the stability of temozolomide in solutions prepared from the commercially available powder for intravenous infusion. The study resulted that more than 90% of temozolomide remained intact after storage for 9 days at room temperature (2.5 mg/mL) and 13 weeks at 5° C. (1.25 mg/mL)

As temozolomide is a cytotoxic drug, the preparation of oral suspensions from capsules or oral solutions from injection pose a high risk for the dispenser or the health professional to directly come in contact with the drug due to spillage of powder from the capsule or liquid from the injection.

The currently marketed formulations are suitable for adults, but they are not adopted for pediatric patients. Indeed, the children find difficulty in swallowing the capsules, and the intravenous administration is quite traumatic. This situation is particularly critical for newborn babies as well as young children. In addition, chemotherapy intravenous administration is not appropriate for ambulatory treatments, wherein the children have to take a daily dose over months.

From the prior art, it is observed that there are issues with the stability of temozolomide when formulated in oral suspension form. Further, due to cytotoxic effect of temozolomide, precaution is required during the handling of temozolomide formulation.

Thus, there is a need, in general, to provide stable pharmaceutical composition of temozolomide, which remains stable during the course of therapy and provides with increasing patient compliance thereby optimizing therapeutic actions of temozolomide.

### Objects of the Invention

The primary object of the present invention is to provide a stable oral pharmaceutical composition comprising temozolomide and one or more pharmaceutically acceptable excipients, wherein the said composition is in the form of a powder for oral suspension.

Another object of the present invention is to provide a stable oral pharmaceutical composition comprising temozolomide and one or more pharmaceutically acceptable excipients, wherein the said composition is in the form or a powder for oral suspension, wherein the said powder is reconstituted with a liquid vehicle just before administration.

Another object of the present invention is to provide a stable oral pharmaceutical composition comprising temozolomide and one or more pharmaceutically acceptable excipients, wherein the said composition is in the form of a powder for oral suspension and comprises of temozolomide, diluent, glidant, stabilizer and optionally a sweetener and flavoring agent.

Another object of the present invention is to provide a stable oral pharmaceutical composition comprising temozolomide and one or more pharmaceutically acceptable excipients, wherein the said composition is in the form of a powder for oral suspension, wherein the said powder is reconstituted with a liquid vehicle just before administration; wherein the said liquid vehicle comprises of suspending agent, one or more solvent, and optionally a sweetener, preservative and a flavoring agent.

Another object of the present invention is to provide a stable oral pharmaceutical composition comprising temozolomide and one or more pharmaceutically acceptable excipients, wherein the said composition is in the form of a powder for oral suspension, wherein the said powder is filled into a device which allows the reconstitution of said powder with a liquid vehicle just before administration.

Another object of the present invention is to provide process for the preparation of a stable oral pharmaceutical composition comprising temozolomide and one or more pharmaceutically acceptable excipients, wherein the said composition is in the form of a powder for oral suspension.

Another object of the present invention is to provide process for the preparation of a stable oral pharmaceutical composition comprising temozolomide and one or more pharmaceutically acceptable excipients, wherein the said composition is in the form of a powder for oral suspension, wherein the said powder is filled in to a device which allows the reconstitution of said powder with a liquid vehicle just before administration.

Another object of the present invention is to provide a stable oral pharmaceutical composition comprising temozolomide and one or more pharmaceutically acceptable excipients, wherein the said composition is in the form of a powder for oral suspension, wherein the said powder is filled in to a device which allows the reconstitution of the said powder and prevents the exposure of cytotoxic effect of temozolomide.

Another object of the present invention is to provide a stable oral pharmaceutical composition comprising temozolomide and one or more pharmaceutically acceptable excipients, wherein the said composition is in the form of a powder for oral suspension, wherein the said powder is filled in to a device which allows the reconstitution of the said powder and masks the bitter taste of temozolomide.

Another object of the present invention is to provide a stable oral pharmaceutical composition comprising temozolomide and one or more pharmaceutically acceptable excipients, wherein the said composition is in the form of a powder for oral suspension, wherein the said suspension is used for administration to pediatric patients.

Another object of the present invention is to provide a stable oral pharmaceutical composition comprising temozolomide and one or more pharmaceutically acceptable excipients, wherein the said composition is in the form or a powder for oral suspension, wherein the said composition does not have more than 5% (w/w) of total impurity, more preferably does not have more than 3% of total impurity of Temozolomide after being stored at specific storage conditions.

### SUMMARY OF THE INVENTION

The present invention provides a stable oral pharmaceutical composition comprising temozolomide and one or more pharmaceutically acceptable excipients, wherein the said composition is in the form of a powder for oral suspension, wherein the said powder is filled into a device which allows the reconstitution of said powder with a liquid vehicle just before administration. Further, the present invention also provides process for the preparation of said pharmaceutical composition comprising temozolomide according to the present invention, and their use in treatment of patients in need thereof.

### DETAILED DESCRIPTION

The detailed description and the examples provided herein are exemplary and any modification or variation within the scope of the invention will be apparent to a person skilled in the art. Further, unless otherwise defined, all the technical and scientific terms used herein shall bear the meaning as understood by a person who is ordinarily skilled in the art.

The present invention provides a stable oral pharmaceutical composition comprising temozolomide and one or more pharmaceutically acceptable excipients, wherein the said composition is in the form of a powder for oral suspension, wherein the said powder is filled into a device which allows the reconstitution of said powder with a liquid vehicle just before administration. Further, the present invention also provides process for the preparation of said pharmaceutical composition comprising temozolomide according to the present invention, and their use in treatment of newly diagnosed glioblastoma multiforme and Refractory Anaplastic Astrocytoma in patients need thereof.

The term “Temozolomide” used throughout the specification refers to their pharmaceutically acceptable salt, pharmaceutically acceptable solvates, pharmaceutically acceptable hydrates, pharmaceutically acceptable enantiomers, pharmaceutically acceptable derivatives, pharmaceutically acceptable polymorphs and pharmaceutically acceptable pro-drugs thereof, wherein the amount of temozolomide is present from 30% w/w to 60% w/w of total composition which is in the powder form of preparation. Further after the reconstitution the amount of temozolomide is present from 5 mg/mL to 50 mg/mL of total composition before administration.

The term “pharmaceutical composition” for the purpose of the invention, means a composition in the form of powder for the preparation of oral suspension, wherein the said powder can be reconstituted with a liquid vehicle just before administration.

The said pharmaceutical composition comprises of temozolomide and one of more pharmaceutically acceptable excipients.

The term “pharmaceutically acceptable” means salt, carriers, excipients, and other formulation ingredients that are compatible with all other pharmaceutical ingredients of a composition and are not deleterious to an individual treated with composition.

The term “specific storage conditions” as used throughout the specification, refers to the pharmaceutical composition of present invention stored for at least one month, more preferably six months at 40° C./75% RH.

The composition of temozolomide is reconstituted with liquid vehicle to obtain suspension of temozolomide just before administration, wherein the said suspension of temozolomide remains stable for at least 7 days at 25° C. and 60% RH and for at least 14 days at 0° C. to 8° C.

The term “total impurities” of temozolomide as used throughout the specification, refers to identified or unidentified degradation product or impurity structurally related with temozolomide which are arising from a manufacturing process or during storage of material.

The term “unspecified impurity” of temozolomide as used throughout the specification refers to can be either any unidentified impurity which is arising from a manufacturing process or during storage of material at specific storage conditions

The term “stable” as used throughout the specification, refers to oral pharmaceutical composition of temozolomide and one or more pharmaceutically acceptable excipients, wherein the said composition is in the form of powder and does not contain more than 5% (w/w) of total impurity. The stable powder for oral suspension refers to a pharmaceutical composition in which the active ingredient, temozolomide, is present in an amount of at least 90% of the original label specified amount for each such ingredient stored at 40° C./75% RH for at least one month, more preferably six months.

The term “powder” as used throughout the specification, refers to either a blend of temozolomide and one or more pharmaceutically acceptable excipients, or granules prepared by mixing of one or more pharmaceutically acceptable excipients or any of the conventional granulation techniques such as wet granulation, dry granulation and direct compaction. The said conventional granulation techniques are well-known in the literature.

The term “stabilizer” used throughout the specification refers to any chemical or agents inhibit the degradation of Temozolomide by inhibiting the formation of a degradation product. The preferred stabilizer used according to the present invention is an organic acid such as ascorbic acid and its derivatives, malic acid, isoascorbic acid, citric acid, tartaric acid, and mixtures thereof.

The term “device” as used throughout the specification, refers to the packaging container comprising two components. In one embodiment, a device comprises a dual-component packaging comprising: (a) a stable oral pharmaceutical composition comprising temozolomide and one or more pharmaceutically acceptable excipients in the first component, (b) a liquid vehicle in the second component. In another embodiment, the device can be a dual-chamber packaging comprising: (a) stable oral pharmaceutical composition comprising temozolomide and one or more pharmaceutically acceptable excipients, (b) a liquid vehicle, wherein the said composition can be reconstituted with the liquid vehicle in the said device just before administration.

Preferably, the said device comprises a dual-chamber bottle comprising: (a) a stable oral pharmaceutical composition comprising temozolomide and one or more pharmaceutically acceptable excipients in the first chamber, (b) a liquid vehicle in the second chamber, wherein the said composition can be reconstituted with the liquid vehicle in the said device just before administration. The dual-chamber bottle comprises first chamber in the bottle cap and the liquid vehicle in the bottle chamber. The dual-chamber bottle of the present invention is designed such that the pharmaceutical composition is filled in the bottle cap chamber and is released into liquid vehicle chamber of the bottle by applying pressure and tightening of the cap, and hence the pharmaceutical composition can be reconstituted with liquid vehicle in the said bottle chamber prior to administration.

One of the embodiments of the present invention is to provide a stable oral pharmaceutical composition comprising temozolomide and one or more pharmaceutically acceptable excipients, wherein the said composition is in the form of a powder for oral suspension.

Another embodiment of the present invention is to provide a stable oral pharmaceutical composition comprising temozolomide and one or more pharmaceutically acceptable excipients, wherein the said composition is in the form or a powder for oral suspension, wherein the said powder is reconstituted with a liquid vehicle just before administration.

Another embodiment of the present invention is to provide a stable oral pharmaceutical composition comprising temozolomide and one or more pharmaceutically acceptable excipients, wherein amount of temozolomide in the said stable powder for oral suspension is in the range from 5 mg/ml to 50 mg/ml, more preferably from 10 mg/ml to 30 mg/ml.

Yet another embodiment of the present invention is to provide a stable oral pharmaceutical composition comprising temozolomide and one or more pharmaceutically acceptable excipients, wherein the said composition is in the form of a powder for oral suspension and comprises of temozolomide, diluent, glidant, stabilizer, and optionally a sweetener and flavoring agent.

Yet another embodiment of the present invention is to provide a stable oral pharmaceutical composition comprising temozolomide and one or more pharmaceutically acceptable excipients, wherein the said composition is in the form or a powder for oral suspension, wherein the said powder is reconstituted with a liquid vehicle just before administration; wherein the said liquid vehicle comprises of suspending agent, one or more solvent, and optionally a sweetener, preservative and a flavoring agent.

Yet another embodiment of the present invention is to provide a stable oral pharmaceutical composition comprising temozolomide and one or more pharmaceutically acceptable excipients, wherein the said composition is in the form of a powder for oral suspension, wherein the said powder is filled into a device which allows the reconstitution of said powder with a liquid vehicle just before administration.

Yet another embodiment of the present invention is to provide process for the preparation of a stable oral pharmaceutical composition comprising temozolomide and one or more pharmaceutically acceptable excipients, wherein the said composition is in the form of a powder for oral suspension.

Yet another embodiment of the present invention is to provide process for the preparation of a stable oral pharmaceutical composition comprising temozolomide and one or more pharmaceutically acceptable excipients, wherein the said composition is in the form of a powder for oral suspension, wherein the said powder is filled in to a device which allows the reconstitution of said powder with a liquid vehicle just before administration.

Yet another embodiment of the present invention is to provide a stable oral pharmaceutical composition comprising temozolomide and one or more pharmaceutically acceptable excipients, wherein the said composition is in the form of a powder for oral suspension, wherein the said powder is filled in to a device which allows the reconstitution of the said powder and prevents the exposure of cytotoxic effect of temozolomide.

Yet another embodiment of the present invention is to provide a stable oral pharmaceutical composition comprising temozolomide and one or more pharmaceutically acceptable excipients, wherein the said composition is in the form of a powder for oral suspension, wherein the said powder is filled in to a device which allows the reconstitution of the said powder and masks the bitter taste of temozolomide.

Yet another embodiment of the present invention is to provide a stable oral pharmaceutical composition comprising temozolomide and one or more pharmaceutically acceptable excipients, wherein the said composition is in the form of a powder for oral suspension, wherein the said suspension is used for administration to pediatric patients.

Yet another embodiment of the present invention is to provide a stable oral pharmaceutical composition comprising temozolomide and one or more pharmaceutically acceptable excipients, wherein the said composition is in the form or a powder for oral suspension, wherein the said composition does not have more than 5% (w/w) of total impurity, more preferably does not have more than 3% of total impurity of temozolomide after being stored at specific storage conditions.

According to the embodiment of the present invention, the pharmaceutical composition of the present invention comprises of a diluent, wherein the said diluent can be selected form the group comprising of lactose, calcium phosphate, calcium sulfate, calcium carbonate, mannitol, xylitol, sucrose, maltose, fructose, dextrose, and maltodextrin or mixtures thereof. The diluents can be present in a concentration of from about 30% to about 60% by weight of the total weight of the composition.

In one of the preferred embodiments, the said pharmaceutical composition of the present invention comprises preferably mannitol as a diluent. Further, mannitol may have a dual role as a diluent and may also act as a sweetener. Also, mannitol is present in a ratio equal to or less than as compared to temozolomide.

According to the embodiment of the present invention, the pharmaceutical composition of the present invention comprises of a stabilizer, wherein the said stabilizer is preferably an organic acid. The said stabilizer is present in a concentration of at least 1% w/w to 5% w/w of the said pharmaceutical composition. More preferably, the said stabilizer is tartaric acid.

According to the embodiment of the present invention, the pharmaceutical composition of the present invention comprises of a glidant, wherein the said glidant is selected from a group comprising of colloidal silicon dioxide, magnesium silicate, starch, talc, tribasic calcium phosphate, stearic acid, palmitic acid or mixtures thereof. The said glidant is present in a concentration of at least 0.5% w/w to 2% w/w of the said pharmaceutical composition. More preferably, the said glidant is colloidal silicon dioxide.

According to the embodiment of the present invention, the liquid vehicle of the present invention comprises of suspending agent, wherein the said suspending agent is selected from a group comprising of carrageenan, cellulose ether, xanthan gum, sodium alginate, microcrystalline cellulose. The suspending agent helps in appropriately suspending the ingredients of the suspension and prevents formation of a cake at the bottom of the device. More preferably, the said suspending agent is microcrystalline cellulose. The said suspending agent is present in a concentration of at least 0.5% w/w to 10% w/w of the said pharmaceutical composition

According to the embodiment of the present invention, the liquid vehicle of the present invention comprises of preservatives, wherein the said preservatives is selected from a group comprising of Alcohol, Benzalkonium Chloride, Benzethonium Chloride, Benzoic Acid, Benzyl Alcohol, Boric Acid, Bronopol, Butylene Glycol, Butylparaben, Calcium Acetate, Calcium Chloride, Calcium Lactate, Cetrimide, CetylpyridiniumChloride, Chlorhexidine, Chlorobutanol, Chlorocresol, Chloroxylenol, Citric Acid Monohydrate, Cresol, Glycerin, Hexetidine, Imidurea, Methylparaben, Monothioglycerol, Phenol, Phenoxyethanol, Phenylethyl Alcohol, Phenylmercuric Acetate, Phenylmercuric Borate, Phenylmercuric Nitrate, Potassium Benzoate, PotassiumMetabisulfite, Potassium Sorbate, Propionic Acid, Propylene Glycol, Propylparaben, Propylparaben Sodium, SodiumAcetate, Sodium Benzoate, Sodium Borate, Sodium Lactate, Sodium Metabisulfite, Sodium Propionate, Sodium Sulfite, Sorbic Acid, Sulfur Dioxide, Thimerosal. More preferably, the said preservatives is methyl paraben and propyl paraben. The said preservative is present in a concentration of at least 0.5% w/w to 5% w/w of the said pharmaceutical composition.

According to the embodiment of the present invention, the liquid vehicle of the present invention comprises of solvent selected from a group of water, hydro-alcoholic, polyhydric alcohols. More preferably, the solvent selected are sorbitol, glycerol, water and mixtures thereof. Sorbitol and glycerol provide dual function as a solvent as well as a sweetener, which helps in masking the taste of temozolomide.

According to the embodiment of the present invention, the sweetener can be selected form the group comprising of alitame, acesulfame potassium, aspartame, D-tryptophan, dextrose, erythritol, fructose, galactose, glycerol, glycyrrhizin, glucose, isomalt, xylitol, xylose, lactitol, lactose, levulose, maltitol, maltodextrin, maltol, maltose, mannitol, corn syrup, neohesperidindihydrochalcone, neotame, saccharin, siclamate, sorbitol, sucralose, sucrose, tagatose, taumatin and trehalose. The said sweetener is present in a concentration of at least 0.1% w/w to 10% w/w of the said pharmaceutical composition.

According to the embodiment of the present invention, the flavoring agent can be selected form the group comprising of essential oils including peppermint oil, orange oil, lemon oil or can be selected from fruit flavors. The flavoring agent can be present in a concentration of from about 0.1% w/w to about 10% w/w of said pharmaceutical composition.

In yet another embodiment, the present invention is to provide a stable pharmaceutical composition comprising temozolomide and one or more pharmaceutically acceptable excipients, wherein the said composition is in the form of a powder for oral suspension, wherein the said powder is reconstituted with a liquid vehicle just before administration; and is used for the treatment of newly diagnosed glioblastoma multiforme and Refractory Anaplastic Astrocytoma in adult as well as pediatric patients.

According to present invention, the process for the preparation of the pharmaceutical composition comprising temozolomide and one or more pharmaceutical acceptable excipients, comprises the following steps:
(a) Mixing Temozolomide, one or more diluent, one or more stabilizer, one or more glidant to obtain a blend.
(b) Optionally adding sweetener and flavoring agent to the blend obtained in step a) to obtain the pharmaceutical composition of temozolomide in powder form for oral suspension.

According to present invention, the process for the preparation of liquid vehicle utilized for the reconstitution of pharmaceutical composition of temozolomide for preparation of oral suspension is as follows:
(a) Dispersing/dissolving the suspending agent along with stirring in solvent.
(b) Optionally adding preservative, sweetener and flavoring agent in step a) along with stirring.
(c) Making up the volume with water along with stirring.

According to present invention, process for the preparation of a stable oral pharmaceutical composition comprising temozolomide and one or more pharmaceutically acceptable excipients, wherein the said composition is in the form of a powder for oral suspension, wherein the said powder is filled in to a device which allows the reconstitution of said powder with a liquid vehicle just before administration, comprises the step of:

For pharmaceutical composition of temozolomide:
(a) Mixing Temozolomide, one or more diluent, one or more stabilizer, one or more glidant to obtain a blend.
(b) Optionally adding sweetener and flavoring agent to the blend obtained in step a) to obtain the pharmaceutical composition of temozolomide in powder form for oral suspension.

For liquid vehicle:
(a) Dispersing or dissolving the suspending agent along with stiffing in solvent.
(b) Optionally adding preservative, sweetener and flavoring agent in step a) along with stirring.
(c) Making up the volume with water along with stiffing.

In yet another embodiment, the present invention is to provide a stable pharmaceutical composition comprising temozolomide and one or more pharmaceutically acceptable excipients, wherein said composition is reconstituted with liquid vehicle to obtain suspension of temozolomide just before administration, wherein the said reconstituted suspension of temozolomide remains stable for at least 7 days at 25° C. and 60% RH and for at least 14 days at 0° C. to 8° C.

The pharmaceutical composition of temozolomide and liquid vehicle are filled in a device. Further, the device allows the reconstitution of pharmaceutical composition of temozolomide and liquid vehicle just before administration.

In yet another embodiment, the present invention is to provide a stable pharmaceutical composition comprising temozolomide and one or more pharmaceutically acceptable excipients, wherein the said composition is in the form of a powder for oral suspension, wherein the said powder is reconstituted with a liquid vehicle just before administration; and the said composition can be used as single dose or multi dose administration formulation in adult as well as pediatric patients.

In order to further illustrate the present invention, the following examples are provided for the purpose of clarity of understanding. However, it is not intended in any way to limit the scope of present invention and it is readily apparent to those of ordinary skill in the art in light of the teachings of this invention that certain changes and modifications may be made thereto without departing from the scope of the invention.

### EXAMPLE—1: General Composition

| Ingredient | % w/w |
|---|---|
| Temozolomide | 30-60% |
| Diluent | 30-60% |
| Stabilizer | 1-5% |
| Glidant | 0.5-2% |
| Sweetener (Optional) | 0.1-10% |
| Flavouring agent (Optional) | 0.1-10% |

Manufacturing Process:

(a) Mixing Temozolomide, one or more diluent, one or more stabilizer, one or more glidant to obtain a blend.

(b) Optionally adding sweetener and flavoring agent to the blend obtained in step a) to obtain the pharmaceutical composition of Temozolomide in powder form for oral suspension.

### EXAMPLE 2: Pharmaceutical Composition of Temozolomide

| | | |
|---|---|---|
| Strengths | 150 mg/ | 300 mg/ |
| Ingredients | 7.5 ml | 15 ml |
| Temozolomide | 150 | 300 |
| Mannitol (Perlitol SD 100) | 135.9 | 271.8 |
| Colloidal Silicon dioxide | 1.5 | 3 |
| Tartaric acid | 10.5 | 21 |
| Sucralose | 1.5 | 3 |
| Flavor (forest Berry) | 0.6 | 1.2 |
| Total Blend | 300 | 600 |
| Liquid Vehicle | | |
| Microcrystalline cellulose | 112.5 | 225 |
| Glycerol | 1125 | 2250 |
| Sorbitol | 3375 | 6750 |
| Water | Qs to 7.5 ml | Qs to 15 ml |
| Packaging component | | |
| Dual Chamber PET bottle | 1 No. | 1 No. |
| Powder filling Kit | 1 No. | 1 No. |

Manufacturing Process:

(a) Mixing Temozolomide, mannitol, colloidal silicon dioxide, tartaric acid to obtain a blend.

(b) Adding sucralose and forest berry flavors to the blend obtained in step a) to obtain the final blend of temozolomide powder.

(c) Dispersing microcrystalline cellulose along with stirring in glycerol.

(d) Adding the sorbitol in the dispersion obtained in step c)

(e) Making the final volume of liquid vehicle with water along with stiffing.

(f) Filling up the temozolomide powder blend of step b) and liquid vehicle of step e) in dual chamber bottle device to final composition of oral suspension.

STABILITY STUDY:

TABLE 1
Stability data of 150 mg/7.5 ml
imatinib powder for solution of example 2
at condition of 40° C./75% RH.

| | Any | |
|---|---|---|
| Stability | unspecified | Total |
| stations | impurity | impurity |
| Initial | ND | 0.035 |
| 3 months | ND | 0.135 |

The above data shows a total impurity not more than 5% of temozolomide in the formulation, indicative of stability of Temozolomide powder for oral suspension in the drug product.

### EXAMPLE—3: Pharmaceutical Composition of Temozolomide (without Preservative—Unit Dose)

| | | |
|---|---|---|
| Strengths | 150 mg/ | 300 mg/ |
| Ingredients | 7.5 ml | 15 ml |
| Temozolomide | 150 | 300 |
| Mannitol (Perlitol SD 100) | 138 | 276 |
| Colloidal silicon di oxide | 1.5 | 3 |
| Tartaric acid | 10.5 | 21 |
| Liquid Vehicle | | |
| Microcrystalline cellulose | 112.5 | 225 |
| Glycerol | 1125 | 2250 |
| Sorbitol | 3375 | 6750 |
| Sucralose | 1.5 | 3 |
| Flavor (forest Berry) | 0.6 | 1.2 |
| Water | Qs to 7.5 ml | Qs to 15 ml |
| Packaging component | | |
| Dual Chamber PET bottle | 1 No. | 1 No. |
| Powder filling Kit | 1 No. | 1 No. |

Manufacturing Process:

(a) Mixing Temozolomide, mannitol, colloidal silicon dioxide, tartaric acid to obtain a blend of Temozolomide powder.

(b) Dispersing microcrystalline cellulose along with stirring in glycerol.

(c) Adding sorbitol, sucralose and forest berry flavors in the dispersion obtained in step b)

(d) Making the final volume of liquid vehicle with water along with stiffing.

(e) Filling up the temozolomide powder blend of step a) and liquid vehicle of step d) in dual chamber bottle device.

STABILITY STUDY:

TABLE 2
Stability data of 150 mg/7.5 ml Temozolomide powder of example
3 at condition of 40° C./75% RH (Without preservative).

| Related | Stability | | 30 | 60 | 90 | 180 |
|---|---|---|---|---|---|---|
| substances | Limits | Initial | Days | Days | Days | Days |
| Unspecified | NMT | ND | ND | ND | ND | ND |
| Impurity | 0.2% | | | | | |
| Total | NMT | 0.063 | 0.100 | 0.045 | 0.124 | 0.537 |
| Impurities | 5% | | | | | |
| Assay | 90-110% | 100.7 | 98.3 | 100.0 | 99.5 | 100.7 |

The above data shows a total impurity not more than 5% of Temozolomide in the formulation and the assay of Temozolomide is in range of 90-110%, indicative of stability of Temozolomide powder.

TABLE 3
Stability data of reconstituted 150 mg/7.5 ml Temozolomide
powder for suspension of example 3 (Without preservative)

| Related | Stability | 25° C./60% RH | | | | | 2-8° C. | | |
|---|---|---|---|---|---|---|---|---|---|
| substances | Limits | Initial | 1 Days | 3 Days | 5 Days | 7 Days | 3 Days | 7 Days | 14 Days |
| Unspecified | NMT | ND | 0.056 | 0.067 | 0.070 | 0.115 | ND | ND | ND |
| Impurity | 0.2% | | | | | | | | |
| Total | NMT | 0.045 | 0.289 | 0.749 | 1.124 | 1.597 | 0.091 | 0.133 | 0.154 |
| Impurities | 5% | | | | | | | | |

The above data shows a total impurity not more than 5% of Temozolomide in the formulation indicative of stability of reconstituted Temozolomide powder for suspension.

EXAMPLE—4: Pharmaceutical Composition of Temozolomide (With Preservative—Unit Dose)

| | | |
|---|---|---|
| Strengths | 150 mg/ | 300 mg/ |
| Ingredients | 7.5 ml | 15 ml |
| Temozolomide | 150 | 300 |
| Mannitol (Perlitol SD 100) | 138 | 271.8 |
| Colloidal Silicon dioxide | 1.5 | 3 |
| Tartaric acid | 10.5 | 21 |
| Total Blend | 300 | 600 |
| Liquid Vehicle | | |
| Microcrystalline cellulose | 112.5 | 225 |
| Glycerol | 1125 | 2250 |
| Sorbitol | 1700 | 3400 |
| Sucralose | 1.50 | 3 |
| Flavor (forest Berry) | 0.60 | 1.2 |
| Methyl paraben | 9.75 | 19.50 |
| Propyl paraben | 2.25 | 4.50 |
| Water | Qs to 7.5 ml | Qs to 15 ml |
| Packaging component | | |
| Dual Chamber PET bottle | 1 No. | 1 No. |
| Powder filling Kit | 1 No. | 1 No. |

Manufacturing Process:

(a) Mixing Temozolomide, mannitol, colloidal silicon dioxide, tartaric acid to obtain a blend.

(b) Dispersing microcrystalline cellulose along with stiffing in glycerol.

(c) Adding the sorbitol, sucralose, methyl paraben, propyl paraben and flavouring agent in the dispersion obtained in step b)

(d) Making the final volume of liquid vehicle with water along with stiffing.

(e) Filling up the temozolomide powder blend of step a) and liquid vehicle of step d) in dual chamber bottle device.

STABILITY STUDY:

TABLE 4
Stability data of reconstituted 150 mg/7.5 ml
Temozolomide powder for suspension
of example 4 at condition of
40° C./75% RH (With preservative).

| Related | Stability | | | | |
|---|---|---|---|---|---|
| substances | Limits | Initial | 30 Days | 60 Days | 90 Days |
| Unspecified | NMT 0.2% | ND | ND | ND | ND |
| Impurity | | | | | |
| Total | NMT 5% | 0.041 | 0.080 | 0.074 | 0.157 |
| Impurities | | | | | |
| Assay | 90-110% | 96.4 | 98.0 | 97.6 | 97.3 |

The above data shows a total impurity not more than 5% of Temozolomide in the formulation, and the assay of Temozolomide is in range of 90-110%, indicative of stability of Temozolomide powder for suspension in the drug product.

The stability data as mentioned above indicate that the pharmaceutical composition comprising temozolomide or its pharmaceutically acceptable salt thereof and one or more pharmaceutical acceptable excipients in powder form reconstituted with a liquid vehicle just before use are stable.

### EXAMPLE—5: Pharmaceutical Composition of Temozolomide (With Preservative—Multi-Dose)

| | | |
|---|---|---|
| Strengths | 100mg/5 mL; | 100mg/5 mL; |
| Ingredients | 40 mL | 80 mL |
| Temozolomide | 800.00 | 1600 |
| Mannitol (Perlitol SD 100) | 736.00 | 1472 |
| Colloidal Silicon dioxide | 8.00 | 16 |
| Tartaric acid | 56.00 | 112 |
| Total Blend | 1600 | 3200 |
| Liquid Vehicle | | |
| Microcrystalline cellulose | 600.00 | 1200 |
| Glycerol | 6000.00 | 12000 |
| Sorbitol | 9066.67 | 18133.34 |
| Sucralose | 8.00 | 16 |
| Flavor (forest Berry) | 3.20 | 6.4 |
| Methyl paraben | 52.00 | 104 |
| Propyl paraben | 12.00 | 24 |
| Water | Qs to 40 ml | Qs to 80 ml |
| Packaging component | | |
| Dual Chamber PET bottle | 1 No. | 1 No. |
| Powder filling Kit | 1 No. | 1 No. |

The oral compositions of the present invention can be administered as multi-dose formulations to pediatrics patients.

1. A pharmaceutical composition of temozolomide comprising (a) a powder blend of temozolomide and pharmaceutically acceptable excipients, (b) a liquid vehicle, wherein the said composition is administered in the form of a powder for oral suspension wherein the said powder is reconstituted with a liquid vehicle just before administration.

2. The oral suspension as claimed in claim 1, wherein said pharmaceutically acceptable excipient are selected from diluent, stabilizer, glidant, optionally sweetener, and flavoring agent or mixtures thereof.

3. The oral suspension as claimed in claim 2, wherein diluent is selected from the group consisting of lactose, calcium phosphate, calcium sulfate, calcium carbonate, mannitol, xylitol, sucrose, maltose, fructose, dextrose, maltodextrin or mixtures thereof

4. The oral suspension as claimed in claim 2, wherein stabilizer is tartaric acid.

5. The oral suspension as claimed in claim 2, wherein glidant is selected from the group consisting of colloidal silicon dioxide, magnesium silicate, starch, talc, tribasic calcium phosphate, stearic acid, palmitic acid or mixtures thereof.

6. The oral suspension as claimed in claim 1, wherein a liquid vehicle comprising suspending agent, solvent, optionally preservative, optionally sweetener or optionally flavoring agent.

7. The oral suspension as claimed in claim 1, wherein the amount of Temozolomide is 30% to 60% of total powder composition.

8. The oral suspension as claimed in claim 1, wherein the said composition packaged as single-dose or multi-dose administration to adults as well as pediatric patients

9. The oral suspension as claimed in claim 1, wherein the said powder does not contain more than 5% of total impurity of temozolomide and assay of temozolomide is in range of 90-110% in the composition, when stored at 40° C./75% RH for at least one months.

10. A process for the preparation of a stable oral pharmaceutical composition comprising temozolomide and one or more pharmaceutically acceptable excipients, wherein the said composition is in the form of a powder for oral suspension, wherein the said powder is filled in to a device which allows the reconstitution of said powder with a liquid vehicle just before administration, comprises the step of:
Mixing Temozolomide, one or more diluent, one or more stabilizer, one or more glidant, optionally one or more sweetener, optionally one or more flavoring agent to obtain a blend.
Dispersing suspending agent along with stirring in solvent
Optionally adding sweetener, preservative, flavouring agent with solvent in the dispersion obtained in step b)
Making the final volume of liquid vehicle with water along with stirring.
Filling up the temozolomide powder blend of step a) and liquid vehicle of step d) in dual chamber bottle device.
